(19) United States
(12) Patent Application Publication
(10) Pub. No.: US 2020/0310538 A1
(43) Pub. Date: Oct. 1, 2020

(54) Method and Brain-computer Interface System for Recognizing Brainwave Signals

(71) Applicant: National Chung-Shan Institute of Science and Technology, Taoyuan City (TW)

(72) Inventors: Po-Lei Lee, Taipei City (TW); Hao-Teng Hsu, Chiayi City (TW); Shyh-Jian Tang, Taoyuan City (TW); Chia-Lung Yeh, Taoyuan City (TW); Yun-Ru Chen, Taoyuan City (TW); Yi-Ju Chiang, Taichung City (TW)

(21) Appl. No.: 16/367,266

(22) Filed: Mar. 28, 2019

(51) Int. Cl. G06F 3/01; A61B 5/0484

(52) U.S. Cl. CPC G06F 3/015; G06F 3/013; A61B 5/04842

(57) ABSTRACT

A method for recognizing brainwave signals of a brain-computer interface (BCI), includes determining a steady-state visual evoked potential (SSVEP) corresponding to an Oz electrode channel according to the brainwave signals when in a rest state; determining the SSVEPs respectively corresponding to the Oz electrode channel, an O1 electrode channel and an O2 electrode channel according to the brainwave signals when in a stimulating state; and determining a stimulus source of a glance attention according to the SSVEPs of the O1 electrode channel and the SSVEP of the O2 electrode channel; wherein the glance attention stimulates the brainwave signals when in the stimulating state.

### BACKGROUND OF THE INVENTION

### 1. Field of the Invention

The present disclosure relates to a method and system for recognizing brainwave signals, and more particularly, to a method and system for recognizing brainwave signals capable of simultaneously recognizing the brain signals for a brain-computer interface.

### 2. Description of the Prior Art

With the development of biomedical technology, facilities for processing physiological signals are widely developed and utilized, e.g. a brain-computer interface (BCI) device utilized for recognizing electroencephalography (EEG) for recording and analysis. In a conventional technology of the BCI utilized for recognizing the electroencephalography, head-mounted devices (HMD) are involved to incorporate with the BCI interface. More specifically, a user may be equipped with the HMD to stare or gaze at a virtual image displayed on a display of the HMD, so as to activate corresponding BCI commands. Practically, a flashing object may be placed on lateral sides of the HMD display. When the user stares or gazes at the flashing object on one of the lateral sides, visual potentials of the electroencephalography are generated accordingly, such that the BCI consequently generates a corresponding command to the visual potentials.

However, when the user stares or gazes at the flashing object on one of the lateral sides of the HMD display to make a command, an attention of the user is distracted from the lateral of the HMD display, such that the user needs to stare back and forth between the HMD display and the lateral sides of the HMD display, which is incapable of reacting to a variation of the HMD display simultaneously. Moreover, a frequent change of gaze attention increases tiredness of the user. Therefore, an improvement is necessary.

### SUMMARY OF THE INVENTION

The present disclosure provides a method and system for recognizing brainwave signals capable of simultaneously recognizing the brainwave signals of a brain-computer interface, so as to avoid the distraction of the attention of the user when activating the commands corresponding to the BCI interface and improve the user scenario.

An embodiment of the present disclosure discloses a method for recognizing brainwave signals of a brain-computer interface (BCI), comprising: determining a steady-state visual evoked potential (SSVEP) corresponding to an Oz electrode channel according to the brainwave signals when in a rest state; determining the SSVEPs respectively corresponding to the Oz electrode channel, an O1 electrode channel and an O2 electrode channel according to the brainwave signals when in a stimulating state; and determining a stimulus source of a glance attention according to the SSVEPs of the O1 electrode channel and the SSVEP of the O2 electrode channel; wherein the glance attention stimulates the brainwave signals when in the stimulating state.

Another embodiment of the present disclosure discloses a brain-computer interface (BCI) system, comprising: an electroencephalography (EEG) device, configured to acquire brainwave signals; and a controller, coupled to the EEG device and configured to determine a steady-state visual evoked potential (SSVEP) corresponding to an Oz electrode channel according to the brainwave signals when in a rest state, determine the SSVEPs respectively corresponding to the Oz electrode channel, an O1 electrode channel and an O2 electrode channel according to the brainwave signals when in a stimulating state, and determine a stimulus source of a glance attention according to the SSVEPs of the O1 electrode channel and the SSVEP of the O2 electrode channel; wherein the glance attention stimulates the brainwave signals when in the stimulating state.

These and other objectives of the present invention will no doubt become obvious to those of ordinary skill in the art after reading the following detailed description of the preferred embodiment that is illustrated in the various figures and drawings.

### BRIEF DESCRIPTION OF THE DRAWINGS

FIG. 1 is a schematic diagram of a brain-computer interface (BCI) system according to an embodiment of the present disclosure.

FIG. 2 is a schematic diagram of an Epoch-average process.

FIG. 3 is a schematic diagram of stimulus sources and a virtual image on a HMD display according to an embodiment of the present disclosure.

FIG. 4 is a schematic diagram of a method according to an embodiment of the present disclosure.

### DETAILED DESCRIPTION

When a user stares at an object, sights perceived by eyes would be transferred to the visual cortex of the brain through the lateral optic nerves and the lateral geniculate nucleus of thalamus. In a brain-computer interface (BCI) system, brainwave-signal electrodes may be respectively placed on positions of an O1 electrode channel, O2 electrode channel and Oz electrode channel on a head of a user, so as to measure evoked potential signals corresponding to the Oz electrode channel, the O1 electrode channel and the O2 electrode channel based on the International 10-20 system.

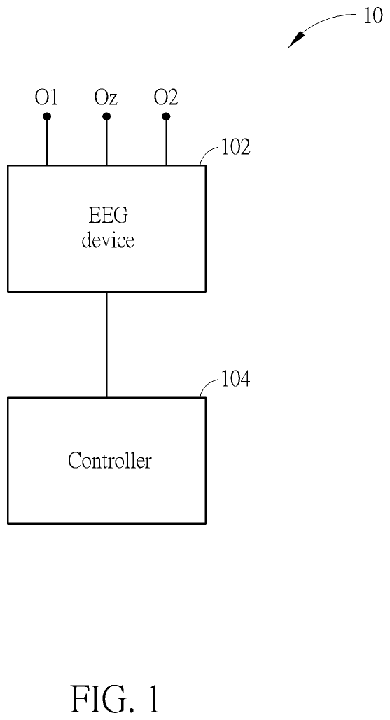

Please refer to FIG. 1, which is a schematic diagram of a brain-computer interface (BCI) system 10 according to an embodiment of the present disclosure. The BCI system 10 includes an electroencephalography (EEG) device 102 and a controller 104. The EEG device 102 is configured to acquire brainwave signals from a user. In an embodiment, the EEG device 102 may record or monitor electrical activities of the brainwave signals of the user. The controller 104 is coupled to the EEG device 102 and configured to determine a steady-state visual evoked potential (SSVEP) corresponding to an Oz electrode channel according to the brainwave signals when in a rest state, determine the SSVEPs respectively corresponding to the Oz electrode channel, an O1 electrode channel and an O2 electrode channel according to the brainwave signals when in a stimulating state, and determine a stimulus source of a gaze attention according to the SSVEPs of the O1 electrode channel and the SSVEP of the O2 electrode channel. That is, the EEG device 102 records the brainwave signals before and after the user is stimulated by a stimulus source, and then the controller 104 determines the SSVEPs corresponding to the O1 electrode channel and the SSVEP of the O2 electrode channel. Therefore, the BCI system 10 of the present disclosure may simultaneously respond to a glance attention made by the user and recognize the brainwave signals.

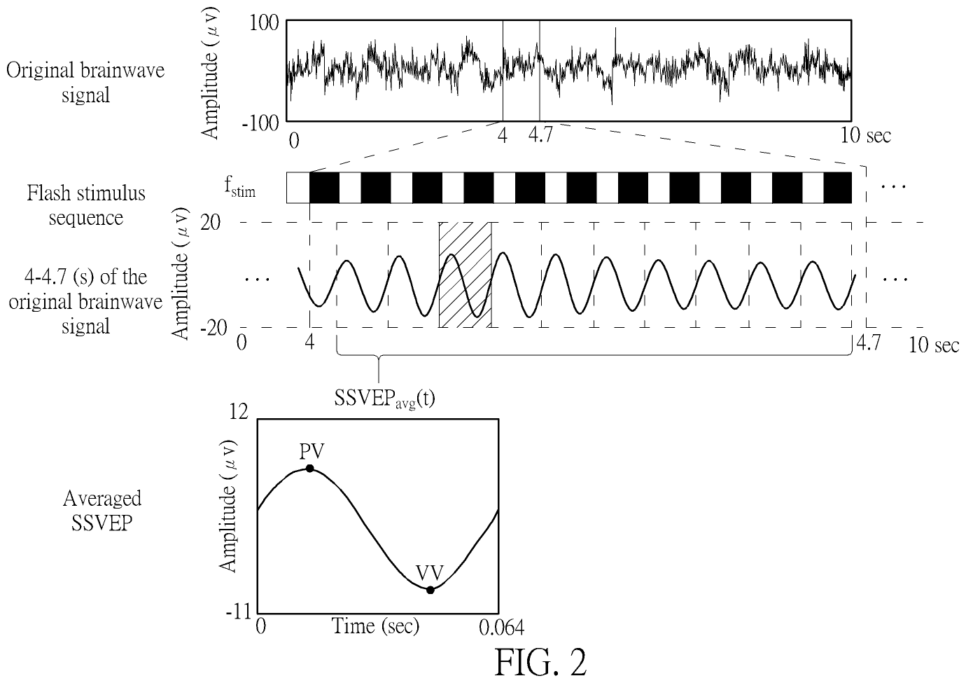

Since the steady state potentials evoked by a lateral view of the user are weaker than that evoked by a direct view, an Epoch-averaged process is applied to increase a signal to noise ratio (SNR) and extract the steady state potentials evoked by the lateral view. Please refer to FIG. 2, which is a schematic diagram of the Epoch-average process. In FIG. 2, an original brainwave signal of the Oz electrode channel for 10 seconds is obtained. A flash stimulus sequence fₛₜᵢₘ with a stimulating flash frequency at 30 Hz is generated based on the original brainwave signal between 4 and 4.7 seconds, which is performed a band-pass filtering between 20 and 31 Hz. The flash stimulus sequence fₛₜᵢₘ consists of a plurality of dark periods and a plurality of bright periods corresponding to the original brainwave signal, and an onset point is defined at a transition of a dark period and a bright period. In an embodiment, a period between two onset points is defined as an Epoch. After the latest 10 Epochs in the flash stimulus sequence fₛₜᵢₘ are determined based on the onset points for averaging, an averaged SSVEP_{avg(t)} is determined. In the example, an amplitude of the averaged SSVEP_{avg(t)} is between −11 microvolt (μv) and 12 μv, and the averaged SSVEP_{avg(t)} is determined from a difference of a maximal amplitude PV and a minimal amplitude VV, which is SSVEP fₛₜᵢₘ=PV−VV (μv).

In an embodiment of the present invention, the brainwave signals of the Oz electrode channel, the O1 electrode channel and the O2 electrode channel are respectively recorded by the EEG 102, such that the controller 104 respectively determines the SSVEPs of the Oz electrode channel, the O1 electrode channel and the O2 electrode channel based on the Epoch-average process.

In an embodiment, the BCI system 10 may further include a head-mounted device (HMD) to display virtual images to the user, such that the user may make commands through the glance attentions. When the user glances at a right side of a HMD display, the brainwave signals (e.g. optic nerve signals) are transferred to a left side of the visual cortex of the brain. In contrast, when the user glances at a left side of the HMD display, the brainwave signals are transferred to a right side of the visual cortex of the brain. In such a situation, the brainwave signals corresponding to different regions of the visual cortex of the brain may be taken as controlling signals of the BCI system 10. Therefore, the BCI system 10 utilizes the glance attention of the user to the stimulus source to determine the commands of the user.

In an operation process of the BCI system 10, the EEG device 102 may record the brainwave signals for a period, e.g. at least 10 seconds, before the user is stimulated or when the bran of the user is in the rest state. The brainwave signals are utilized for determining the SSVEP of the Oz electrode channel by the controller 104 based on the Epoch-average process. Notably, the glance attention of the user triggers/activates the brainwave signals when in the stimulating state. In an embodiment, different flash stimulus sequences at different stimulating flash frequencies may be determined or classified, which may correspond to different stimuli, e.g. different regions of the lateral HMD display respectively correspond to different stimuli glanced by the user. In addition, a threshold related to a maximum of the SSVEP of the Oz electrode channel when in the rest state is determined to determine whether the SSVEP of the O1 electrode channel is greater than the SSVEP of the O2 electrode channel when the SSVEP of the Oz electrode channel is greater than the threshold.

When the SSVEP of the O1 electrode channel is greater than the SSVEP of the O2 electrode channel in the stimulating state, the controller 104 determines that the stimulus source of the glance attention is towards the right side of the HMD display, which represents a command made by the user. Similarly, when the SSVEP of the O1 electrode channel is lower than the SSVEP of the O2 electrode channel in the stimulating state, the controller 104 determines that the stimulus source of the glance attention is towards the left side of the HMD display. Therefore, when the user operates the BCI system 10, the user is free from gazing at the corresponding region of the lateral HMD display to make commands. Instead, the user focuses on the HMD display and glances at the lateral region of the HMD display to make commands for the BCI system 10.

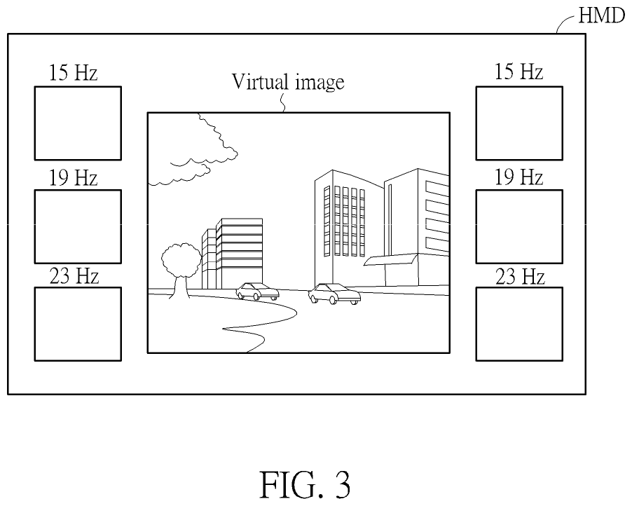

Moreover, the BCI system 10 of the present disclosure may be implemented in all kinds of ways. Since the different flash stimulus sequences at different stimulating flash frequencies correspond to different stimulus sources on the HMD display, the different regions of the lateral HMD display may correspond to different commands. Please refer to FIG. 3, which is a schematic diagram of the stimulus sources and the virtual image on the HMD display according to an embodiment of the present disclosure. As shown in FIG. 3, each of the stimulating flash frequencies corresponds to a region on the lateral HMD display glanced by the user. In a user scenario, the user with the HMD operates with the BIC system 10 and makes commands to move a wheelchair. After the controller 104 determines the SSVEPs of the Oz electrode channel, the O1 electrode channel and the O2 electrode channel before and after the stimulating state, the controller 104 determines whether the SSVEP of the O1 electrode channel is greater than the SSVEP of the O2 electrode channel to determine the stimulus source of the glance attention made by the user. The stimulating flash frequency at 15 Hz of the right side of the HMD display may correspond to a command of “turn right” in the BCI system 10, the stimulating flash frequency at 15 Hz of the left side of the HMD display may correspond to a command of “turn left” in the BCI system 10, the stimulating flash frequency at 19 Hz of the right side of the HMD display may correspond to a command of “move forward” in the BCI system 10, and the stimulating flash frequency at 19 Hz of the left side of the HMD display may correspond to a command of “move back” in the BCI system 10. Similarly, the stimulating flash frequency at 19 Hz of the right side and the left side of the HMD display respectively correspond to different commands in the BCI system 10.

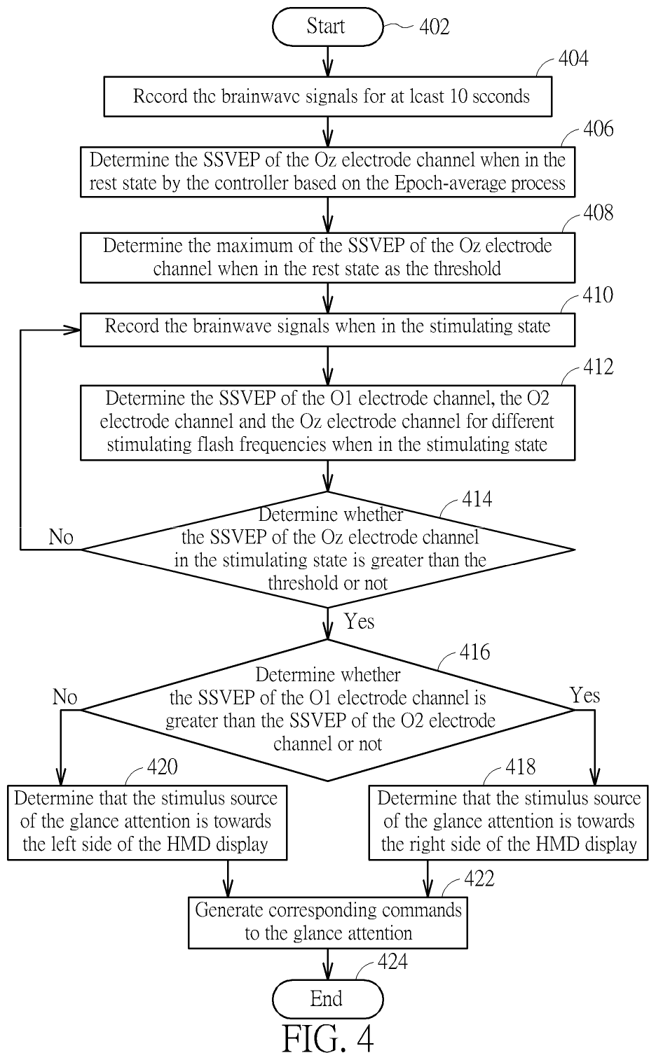

An operation process of the BCI system 10 may be summarized to a method 40 for recognizing the brainwave signals of the BCI system 10 as shown in to FIG. 4, which is a schematic diagram of a method 40 according to an embodiment of the present disclosure. The method 40 includes the following steps:

Step 402: Start.

Step 404: Record the brainwave signals for at least 10 seconds.

Step 406: Determine the SSVEP of the Oz electrode channel when in the rest state by the controller 104 based on the Epoch-average process.

Step 408: Determine the maximum of the SSVEP of the Oz electrode channel when in the rest state as the threshold.

Step 410: Record the brainwave signals when in the stimulating state.

Step 412: Determine the SSVEP of the O1 electrode channel, the O2 electrode channel and the Oz electrode channel for different stimulating flash frequencies when in the stimulating state.

Step 414: Determine whether the SSVEP of the Oz electrode channel in the stimulating state is greater than the threshold or not. If yes, execute step 416; if no, execute step 410.

Step 416: Determine whether the SSVEP of the O1 electrode channel is greater than the SSVEP of the O2 electrode channel or not. If yes, execute step 418; if no, execute step 410.

Step 418: Determine that the stimulus source of the glance attention is towards the right side of the HMD display.

Step 420: Determine that the stimulus source of the glance attention is towards the left side of the HMD display.

Step 422: Generate corresponding commands to the glance attention.

Step 424: End.

The operation of the method 40 can be known by referring to the embodiments of the BCI system 10 described above, and are not repeated herein for brevity.

Notably, the embodiments stated above illustrate the concept of the present disclosure, those skilled in the art may make proper modifications accordingly, and not limited thereto. For example, the different regions of the lateral HMD display may respectively correspond to different commands of the BCI system, flash stimulus sequences at different stimulating flash frequencies may be determined to correspond different stimulus sources on the HMD display, and not limited thereto.

In summary, the present disclosure provides a method and system for recognizing brainwave signals to simultaneously recognize the brainwave signals of the BCI system, which avoids the distraction of the attention of the user when activating the commands corresponding to the BCI interface. Instead, the present disclosure recognizes the brainwave signals corresponding to the glance attention of the user to generate corresponding commands in the BCI system and improve the user scenario.

Those skilled in the art will readily observe that numerous modifications and alterations of the device and method may be made while retaining the teachings of the invention. Accordingly, the above disclosure should be construed as limited only by the metes and bounds of the appended claims.

What is claimed is:

1. A method for recognizing brainwave signals of a brain-computer interface (BCI), comprising:
determining a steady-state visual evoked potential (SSVEP) corresponding to an Oz electrode channel according to the brainwave signals when in a rest state;
determining the SSVEPs respectively corresponding to the Oz electrode channel, an O1 electrode channel and an O2 electrode channel according to the brainwave signals when in a stimulating state; and
determining a stimulus source of a glance attention according to the SSVEPs of the O1 electrode channel and the SSVEP of the O2 electrode channel;
wherein the glance attention stimulates the brainwave signals when in the stimulating state.

2. The method of claim 1, wherein the step of determining the stimulus source of the glance attention according to the SSVEPs of the O1 electrode channel and the SSVEP of the O2 electrode channel comprises:
determining whether the SSVEP of the O1 electrode channel is greater than the SSVEP of the O2 electrode channel when the SSVEP of the Oz electrode channel is greater than a threshold.

3. The method of claim 2, wherein the threshold is related to a maximum of the SSVEP of the Oz electrode channel when in the rest state.

4. The method of claim 2, further comprising when the SSVEP of the O1 electrode channel is greater than the SSVEP of the O2 electrode channel when in the stimulating state, determining that the stimulus source of the glance attention is towards a right side.

5. The method of claim 2, further comprising when the SSVEP of the O1 electrode channel is lower than the SSVEP of the O2 electrode channel when in the stimulating state, determining that the stimulus source of the glance attention is towards a left side.

6. The method of claim 1, wherein the SSVEP is determined based on an Epoch-averaged process.

7. A brain-computer interface (BCI) system, comprising:
an electroencephalography (EEG) device, configured to acquire brainwave signals; and
a controller, coupled to the EEG device and configured to determine a steady-state visual evoked potential (SSVEP) corresponding to an Oz electrode channel according to the brainwave signals when in a rest state, determine the SSVEPs respectively corresponding to the Oz electrode channel, an O1 electrode channel and an O2 electrode channel according to the brainwave signals when in a stimulating state, and determine a stimulus source of a glance attention according to the SSVEPs of the O1 electrode channel and the SSVEP of the O2 electrode channel;
wherein the glance attention stimulates the brainwave signals when in the stimulating state.

8. The BCI system of claim 7, wherein the controller determines whether the SSVEP of the O1 electrode channel is greater than the SSVEP of the O2 electrode channel when the SSVEP of the Oz electrode channel is greater than a threshold.

9. The BCI system of claim 8, wherein the threshold is related to a maximum of the SSVEP of the Oz electrode channel when in the rest state.

10. The BCI system of claim 8, wherein the controller determines that the stimulus source of the glance attention is towards a right side when the SSVEP of the O1 electrode channel is greater than the SSVEP of the O2 electrode channel when in the stimulating state.

11. The BCI system of claim 8, wherein the controller determines the stimulus source of the glance attention is towards a left side when the SSVEP of the O1 electrode channel is lower than the SSVEP of the O2 electrode channel when in the stimulating state.

12. The BCI system of claim 7, wherein the SSVEP is determined based on an Epoch-averaged process.
